(19) United States
(12) Patent Application Publication
(10) Pub. No.: US 2016/0000369 A1
(43) Pub. Date: Jan. 7, 2016

(54) MEASUREMENT DEVICE FOR ASSESSING KNEE MOVEMENT

(71) Applicant: UNIVERSITY OF LOUISVILLE RESEARCH FOUNDATION, INC., Louisville, KY (US)

(72) Inventor: Franky Lydale Minnifield, Lexington, KY (US)

(21) Appl. No.: 14/768,900

(22) PCT Filed: Feb. 24, 2014
(86) PCT No.: PCT/US2014/017944

§ 371 Date: Aug. 19, 2015

Related U.S. Application Data
(60) Provisional application No. 61/769,504, filed on Feb. 26, 2013.
(60) Provisional application No. 61/817,029, filed on Apr. 29, 2013.

(51) Int. Cl. A61B 5/00; A61B 5/11

(52) U.S. Cl. CPC A61B 5/4585; A61B 5/1121

(57) ABSTRACT

A measurement device for assessing knee movement comprises: an elongated base member; a moveable (or sliding) member configured for movement along and relative to the elongated base member; and indicia on an upper surface of the elongated base member to reflect the relative position of the moveable member with respect to the elongated base member. The measurement device may also include multiple blocks arranged in a stack on the elongated base member to measure knee extension of a patient.

### CROSS-REFERENCE TO RELATED APPLICATIONS

The present application claims priority to U.S. patent application Ser. No. 61/769,504 filed on Feb. 26, 2013 and U.S. patent application Ser. No. 61/817,029 filed on Apr. 29, 2013, the entire disclosures of which are incorporated herein by reference.

### BACKGROUND OF THE INVENTION

The present invention relates to a measurement device for assessing knee movement, for example prior to or after a knee surgery or a knee replacement.

For instance, knee-related injuries are one of the most common injuries in sports. Many knee injuries result in a ruptured or torn anterior cruciate ligament (ACL), one of the four major ligaments of the knee. Injury to the ACL is often remedied by reconstructive surgery, followed by several months of physical therapy and rehabilitation. One of the biggest factors that determines whether a patient achieves full recovery is adherence to physical therapy schedules and rehabilitation exercises. Many of these exercises focus on restoring the range-of-motion of the knee. So, such exercises require constant measurement of the range-of-motion of the knee and monitoring of progress made during rehabilitation.

Although there are some complicated devices that exist in the art for measuring the range of motion of a knee, such as goniometers, most are complex mechanical or electromechanical devices that can only be used in a clinical setting with the assistance of a therapist.

Thus, there remains a need for a device that allows for ready assessment of knee movement, which can be used as part of a physical therapy and rehabilitation program, especially when a patient is alone during rehabilitation.

### SUMMARY OF THE INVENTION

The present invention is a measurement device for assessing knee movement, for example prior to or after a knee surgery or a knee replacement. The measurement device generally comprises: an elongated base member; a moveable (or sliding) member configured for movement along and relative to the elongated base member; and indicia on an upper surface of the elongated base member to reflect the relative position of the moveable member with respect to the elongated base member.

In some embodiments, the elongated base member is formed of a first base member and a second base member, which are arranged end-to-end and connected by a hinge. The hinge allows the measurement device to be readily manipulated between an open position and a closed position.

In some embodiments, the measurement device also includes a removable stop. The stop is attached to the elongated base member at a distal end thereof, but it also can be readily removed from the elongated base member.

The moveable member is configured for movement along and relative to the elongated base member. In some embodiments, the moveable member has a generally C-shape, with a main body portion that rests on the upper surface of the elongated base member and two legs that extend along the sides of the elongated base member and engage the elongated base member.

To measure knee flexion, a patient sits down and positions his or her leg on the measurement device in a starting position with the heel of the patient's foot positioned on the elongated base member. The patient begins bending his or her knee, such that the foot slides along the upper surface of the elongated base member. The moveable member is either pushed back by the foot as it slides along the upper surface of the elongated base member, or the moveable member is manually slid along the upper surface of the elongated base member as the patient bends his or her knee. Based on the indicia on the upper surface of the elongated base member, the patient is provided with a ready visual indication of how far the knee can be bent.

In some embodiments, the exemplary measurement device further includes multiple blocks arranged in a stack and positioned at a distal end of the elongated base member. Each of these blocks has a predetermined height and is configured such that the blocks can be stacked on top of one another.

To measure knee extension, a patient sits down and positions his or her leg on the elongated base member of the measurement device. The patient or an attending therapist then stacks one or more blocks on the distal end of the elongated base member and positions the heel of the patient on top of the stack. If the calf of the patient's leg is touching the upper surface of the elongated base member, another block is added to the stack, and the heel of the patient is placed back on top of the stack. This is repeated until the calf of the patient's leg comes off of the elongated base member of the measurement device. This allows for visualization and assessment of the extension of the knee based on the number of blocks in the stack at the time the patient's calf no longer touches the upper surface of the elongated base member.

### DESCRIPTION OF THE DRAWINGS

FIG. 1 is a perspective view of an exemplary measurement device made in accordance with the present invention in an open position;

FIG. 1A is a perspective view of the exemplary measurement device of FIG. 1 as it transitions from the open position to a closed position (or vice versa), and with the stop detached from the elongated base member;

FIG. 2 is an end view of the exemplary measurement device of FIG. 1 with the stop removed;

FIG. 3 is a perspective view of the exemplary measurement device of FIG. 1 in use, with a patient's leg in a starting position;

FIG. 3A is a perspective view identical to FIG. 3, but with the patient bending the knee;

FIG. 4 is a side view of the exemplary measurement device of FIG. 1 shown with multiple blocks arranged in a stack and being used to measure knee extension of a patient in accordance with the present invention; and

FIG. 4A is a side view of the exemplary measurement device of FIG. 4 shown with additional blocks arranged in a stack and being used to measure knee extension of a patient in accordance with the present invention.

### DETAILED DESCRIPTION OF THE INVENTION

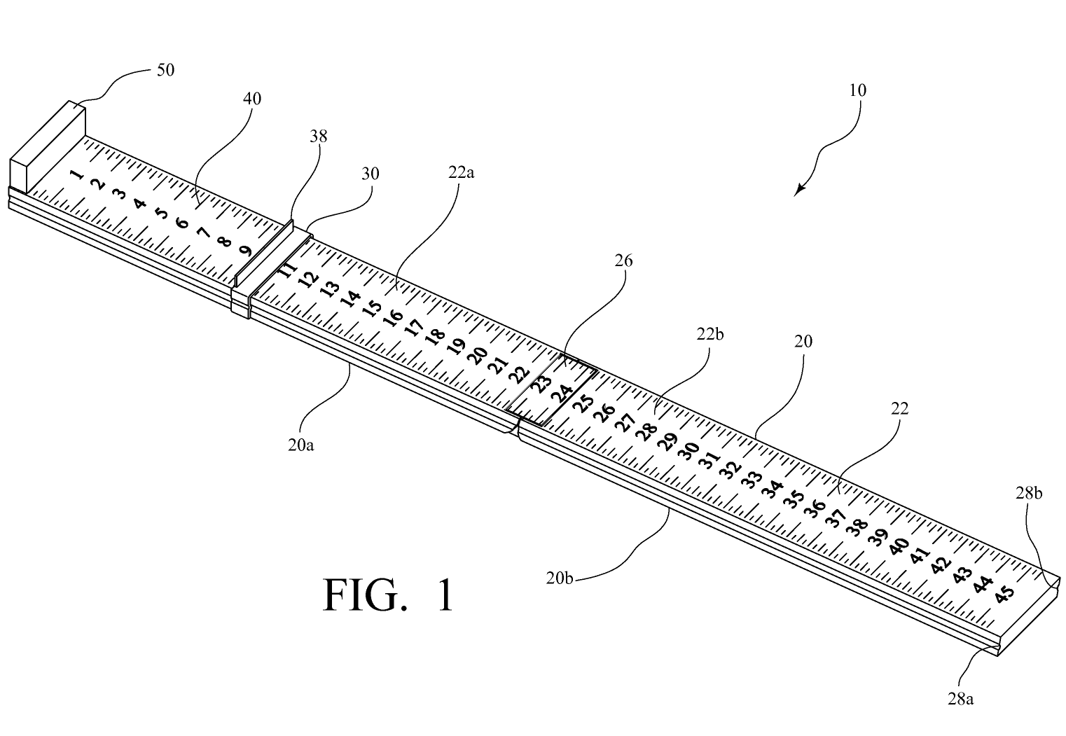

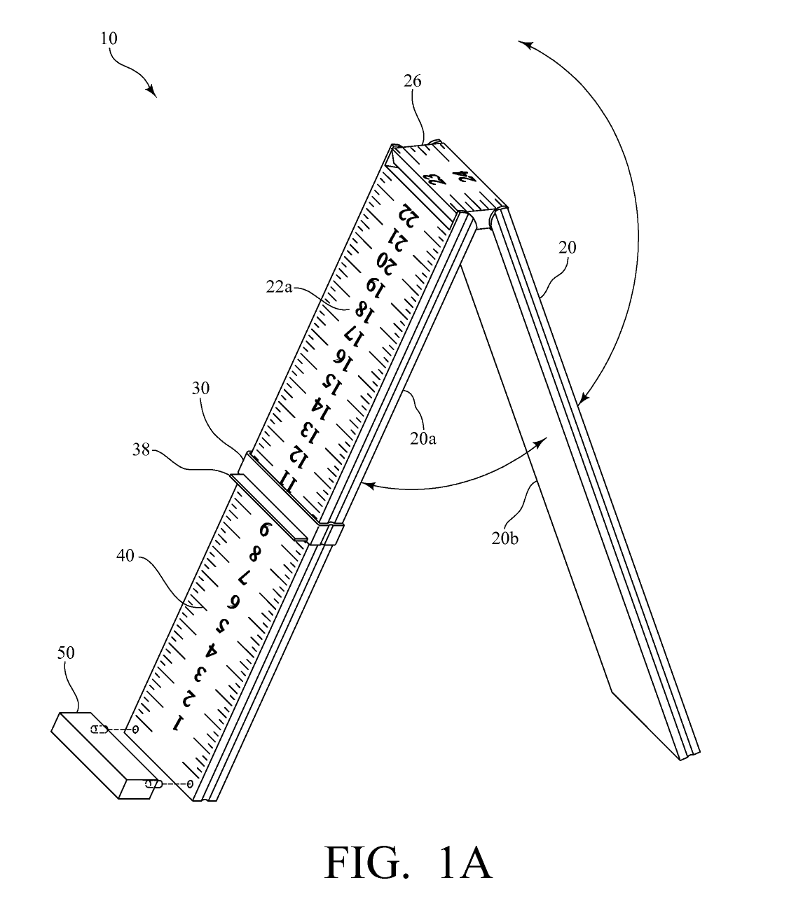

The present invention is a measurement device for assessing knee movement, for example prior to or after a knee surgery or a knee replacement. As shown in FIGS. 1 and 1A, an exemplary measurement device 10 made in accordance with the present invention generally comprises: an elongated base member 20; a moveable (or sliding) member 30 configured for movement along and relative to the elongated base member 20; and indicia 40 on an upper surface 22 of the elongated base member 20 to reflect the relative position of the moveable member 30 with respect to the elongated base member 20.

Referring still to FIGS. 1 and 1A, in this exemplary embodiment, the elongated base member 20 is formed of a first base member 20a and a second base member 20b, which are arranged end-to-end and connected by a hinge 26. The hinge 26 allows the measurement device 10 to be readily manipulated between an open position and a closed position. In other words, the second base member 20b is capable of rotating approximately 180 degrees relative to the first base member 20a about the hinge 26 (as shown by the arrows in FIG. 1A) between the open position and the closed position.

When the measurement device 10 is in the open position, as shown in FIG. 1, the first and second base members 20a, 20b are aligned such that an upper surface 22a of the first base member 20a and an upper surface 22b of the second base member 20b form a substantially continuous upper surface 22 of the elongated base member 20. When the measurement device 10 is in the closed position, the bottom surfaces of the first and second base members 20a, 20b are in contact with each other along their lengths. In this regard, it is contemplated that the overall length of the measurement device 10 in the open position would be approximately four feet, but, in the closed position, the overall length would be reduced to approximately two feet for better portability.

Referring still to FIGS. 1 and 1A, in this exemplary embodiment, the measurement device 10 also includes a stop 50. The stop 50 is attached to the elongated base member 20 at a distal end thereof, but it also can be readily removed from the elongated base member 20, as shown in FIG. 1A. As shown in FIG. 1A, the attachment of the stop 50 to the elongated base member 20 can be facilitated by the use of pegs extending from the bottom of the stop 50 into corresponding holes defined in the upper surface 22 of the elongated base member 20. However, this is but one example of how the stop 50 could be attached to the elongated base member 20, and various other techniques could be used without departing from the spirit and scope of the present invention.

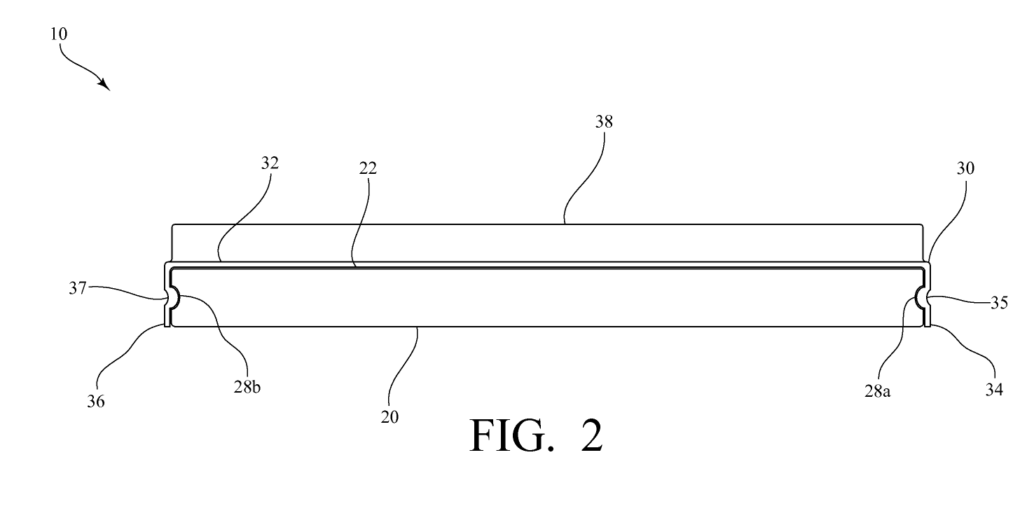

Referring now to FIG. 2, the moveable member 30 is configured for movement along and relative to the elongated base member 20. In this exemplary embodiment, the moveable member 30 has a generally C-shape, with a main body portion 32 that rests on the upper surface 22 of the elongated base member 20. The moveable member 30 also includes two legs 34, 36 that extend along the sides of the elongated base member 20. There are one or more perpendicular extensions (or ridges) 35 on the inside of the first leg 34 and one or more perpendicular extensions (or ridges) 37 on the inside of the second leg 36. Such perpendicular extensions (or ridges) 35, 37 are configured to engage and ride in respective grooves 28a, 28b defined along the sides of the elongated base member 20, thus facilitating sliding movement of the moveable member 30 with respect to the elongated base member 20. However, this is but one example of how the moveable member 30 could be mounted and configured for movement along and relative to the elongated base member 20, and various other techniques for providing such relative movement could be used without departing from the spirit and scope of the present invention.

Referring still to FIG. 2, in this exemplary embodiment, the moveable member 30 also includes a vertical tab 38 that extends from the main body portion 32 away from the upper surface 22 of the elongated base member 20, the importance of which will be further described below.

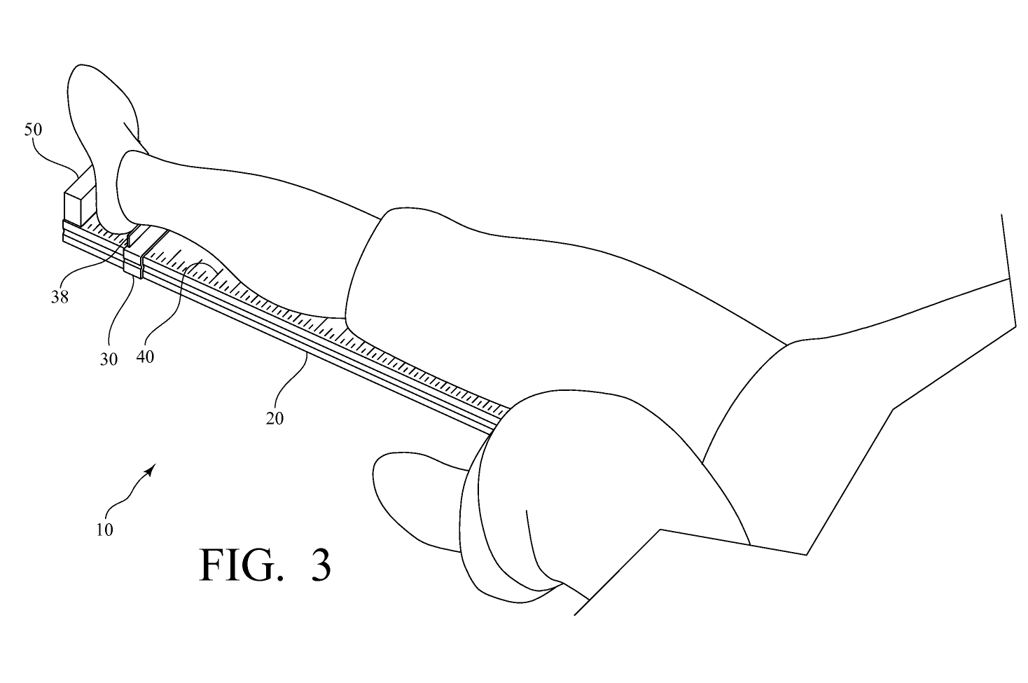

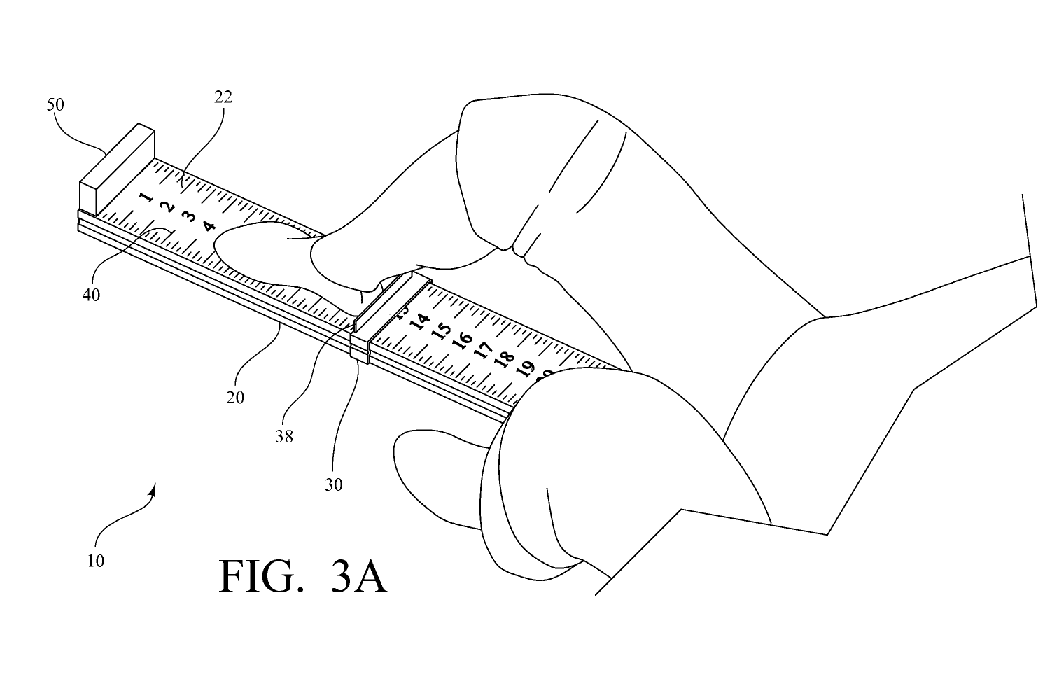

FIGS. 3 and 3A are perspective views of the exemplary measurement device 10 as used to measure knee flexion of a patient. Referring first to FIG. 3, to measure knee flexion, a patient sits down and positions his or her leg on the measurement device 10 in a starting position with the heel of the patient's foot positioned on the elongated base member 20 and resting against the stop 50. The moveable member 30 is then placed directly behind the heel of the patient's foot. This is effectively a “zero position.” Referring now to FIG. 3A, the patient begins bending his or her knee, such that the foot slides back along the upper surface 22 of the elongated base member 20. The moveable member 30 is either pushed back by the foot as it slides along the upper surface 22 of the elongated base member 20, or the moveable member 30 is manually slid along the upper surface 22 of the elongated base member 20 as the patient bends his or her knee. Based on the indicia 40 on the upper surface 22 of the elongated base member 20, the patient is provided with a ready visual indication of how far the knee can be bent. For example, in the first week of therapy, the patient may only be able to advance the moveable member 30 a few inches (or other units) along the elongated base member 20. However, following weeks of rehabilitation, the patient should be able to advance the moveable member 30 further along the elongated base member 20 and will be able to see that improvement.

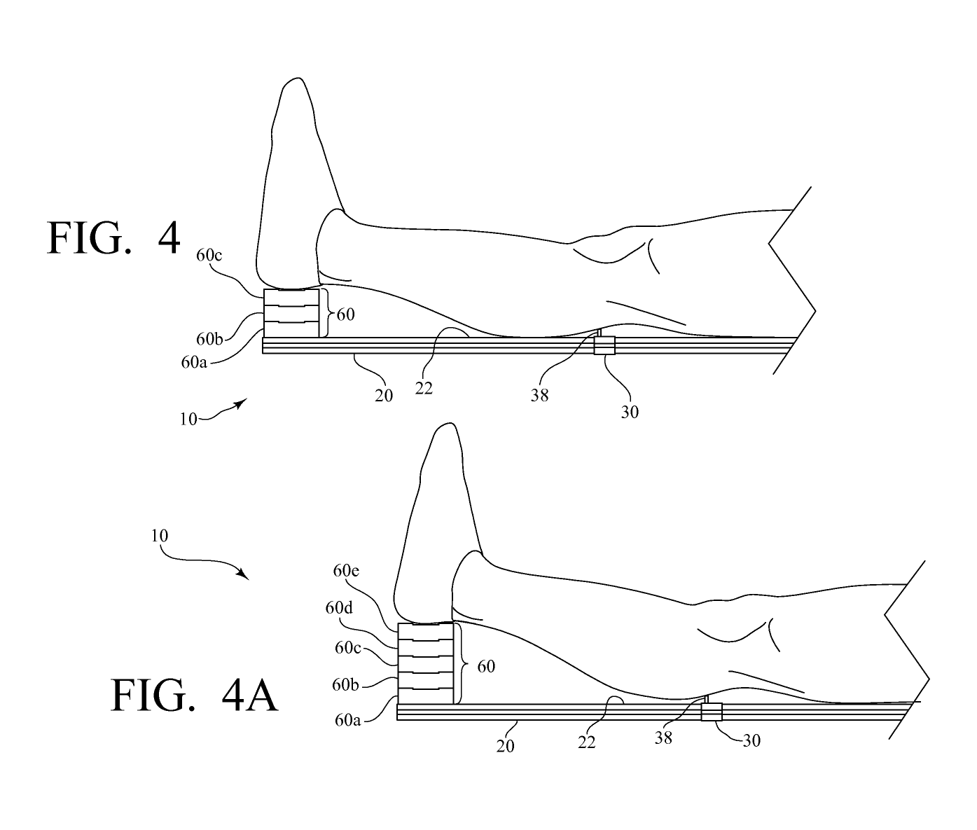

FIGS. 4 and 4A are side views of the exemplary measurement device 10 in which an additional feature is incorporated into the measurement device 10.

FIG. 4A is a side view of the exemplary measurement device of FIG. 4 shown with additional blocks and being used to measure knee extension of a patient in accordance with the present invention. Specifically, as shown in FIGS. 4 and 4A, the measurement device 10 includes multiple blocks 60a-e arranged in a stack 60 and positioned at the distal end of the elongated base member 20 in place of the stop 50. Each of these blocks 60a-e has a predetermined height and is configured such that the blocks 60a-e can be stacked on top of one another. For example, in FIG. 4, three blocks 60a-60c are shown, while in FIG. 4A, five blocks 60a-60e are shown. Furthermore, in FIGS. 4 and 4A, each of the blocks 60a-60e is illustrated as having a projection on a lower surface and a corresponding cavity on an upper surface, so that adjacent blocks 60a-60e can be snapped together in an interference fit. That being said, how the blocks 60a-60e are stacked and secured to one another is not critical to the present invention. Various other techniques could be used to secure the blocks 60a-60e to one another, including, for example, pegs or other fasteners, without departing from the spirit and scope of the present invention. The important point is that the number of blocks 60a-60e, and thus the height of the stack 60, can be varied by a patient or an attending therapist.

Referring still to FIGS. 4 and 4A, to measure knee extension, a patient sits down and positions his or her leg on the elongated base member 20 of the measurement device 10. The patient or the attending therapist then stacks one or more blocks 60a-60e on the distal end of the elongated base member 20 and positions the heel of the patient on top of the stack 60. The position of the patient's leg in relation to the upper surface 22 of the elongated base member 20 is assessed, and if the calf of the patient's leg is touching the upper surface 22 of the elongated base member 20, another block is added to the stack 60, and the heel of the patient is placed back on top of the stack 60. This is repeated until the calf of the patient comes off of the elongated base member 20 of the measurement device 10, as shown in FIG. 4A. This allows for visualization and assessment of the extension of the knee based on the number of blocks in the stack 60 at the time the patient's calf no longer touches the upper surface of the elongated base member 20.

Referring still to FIGS. 4 and 4A, other criteria to determine the extension of the knee are also possible. For example, another way to use the measurement device 10 includes positioning the moveable member 30 under the knee of the patient when the patient's heel is on top of the stack 60. Blocks 60a-60e are then stacked until the back area of the knee of the patient is only slightly touching the vertical tab 38 of the moveable member 30. Alternatively, both the position of the patient's calf relative to the upper surface 22 of the elongated base member 20 and the position of the back area of the knee of the patient relative to the vertical tab 38 of the moveable member 30 may be used for visualization and assessment of the extension of the knee based on the number of blocks 60a-60e in the stack 60.

Although the blocks 60a-e are shown and described as being used in place of the stop 50, it is also contemplated that one more blocks 60a-e could remain on the elongated base member 20 and used as the stop 50 when the measurement device 10 is used to measure the flexion of the patient's knee.

As should be clear from the above discussion, irrespective of the particular construction details, the measurement device of the present invention thus allows for ready visualization and assessment of knee movement, especially when a patient is alone during rehabilitation. Not only does such visualization help the patient see where he/she is in the rehabilitation process, but such visualization can also provide motivation. In other words, since the patient can see progress, the patient can challenge himself or herself to do better each and every day.

Furthermore, the measurement device of the present invention can be tied to a certain protocol or schedule established by a therapist. For example, a therapist could develop an optimal schedule of where the patient should be on a day-by-day basis and then challenge the patient to meet those goals. Indeed, with respect to the bending of the knee and use of the moveable member, the therapist could even mark targets on the device itself near the indicia, so the patient can readily visualize those targets during rehabilitation.

As a further refinement, although not shown in the Figures, it is contemplated that the measurement device of the present invention could include a rubber layer or similar skid-resistant layer that is secured to the bottom surface of the elongated base member so that the measurement device remains stable on an underlying ground surface in use.

As a further refinement, although not shown in the Figures, it is contemplated that the measurement device of the present invention could include a microprocessor and a display screen that would provide real-time feedback as to the position of the moveable member relative to the elongated base member. With respect to the display screen, an LCD screen or similar device could be mounted, for example, on the moveable member for ready viewing by the patient. With respect to the method of use shown in FIGS. 4 and 4A, similar displays could be provided to reflect the number of blocks (or the height of the stack) that can be successfully positioned under the heel.

As a further refinement, although not shown in the Figures, it is also contemplated that the measurement device could include a microprocessor and speaker that would provide audible feedback as to the position of the moveable member relative to the elongated base member.

As a further refinement, it is also contemplated that the measurement device could include a microprocessor and memory register that would log data with respect to the use of the measurement device. Such data would preferably include the results of each measurement, i.e., when the measurement was taken and how far the patient was able to advance the moveable member and/or the number of blocks that were stacked under the heel. The data logs could then be accessed by or transmitted to a therapist for monitoring and review.

As a further refinement, it is also contemplated that the measurement device could be used as part of a system including a data entry device (such as a smart phone hosting a software app) connected to the Internet. A patient or attending therapist would log data about the use of the measurement device using the data entry device. Such data could include, for example, a serial number associated with and distinct to the measurement device, the patient's lower leg length, the patient's upper leg length, the type of injury sustained, and the results of each measurement, i.e., when the measurement was taken and how far the patient was able to advance the moveable member and/or the number of blocks that were stacked under the heel. The data logs could then be stored and subsequently be accessed by or transmitted to a third party over the Internet for review.

Of course, some of the above refinements could be combined in the same measurement device. For example, the measurement device could include a microprocessor and memory register that would log data with respect to the use of the measurement device and automatically communicate the data logs to a data entry device connected to the Internet. In such an embodiment, the measurement device would further include WiFi, Bluetooth®, or similar wireless communication functionality.

One of ordinary skill in the art will recognize that additional embodiments are also possible without departing from the teachings of the present invention. This detailed description, and particularly the specific details of the exemplary embodiment disclosed therein, is given primarily for clarity of understanding, and no unnecessary limitations are to be understood therefrom, for modifications will become obvious to those skilled in the art upon reading this disclosure and may be made without departing from the spirit or scope of the invention.

What is claimed is:

1. A measurement device for assessing knee movement, comprising:
an elongated base member;
a moveable member configured for movement along and relative to the elongated base member; and
indicia on an upper surface of the elongated base member to reflect a relative position of the moveable member with respect to the elongated base member.

2. The measurement device as recited in claim 1, and further comprising a stop attached to the elongated base member at a distal end of the elongated base member.

3. The measurement device as recited in claim 2, wherein the stop is comprised of multiple blocks, each of said multiple blocks having a predetermined height, and each of said multiple blocks being configured to be stacked on top of one another.

4. The measurement device as recited in claim 1, and further comprising multiple blocks for assessing extension of the knee, said multiple blocks located at a distal end of the elongated base member, each of said multiple blocks having a predetermined height, and each of said multiple blocks being configured to be stacked on top of one another.

5. The measurement device as recited in claim 1, wherein the elongated base member is comprised of a first base member and a second base member that are arranged end-to-end.

6. The measurement device as recited in claim 5, wherein the first base member and the second base member are connected by a hinge.

7. A measurement device for assessing post-operative knee movement, comprising:
an elongated base member; and
multiple blocks for assessing extension of the knee, each of said multiple blocks having a predetermined height, and each of said multiple blocks being configured to be stacked on top of one another at a distal end of the elongated base member.

8. The measurement device as recited in claim 7, and further comprising:
a moveable member configured for movement along and relative to the elongated base member; and
indicia on an upper surface of the elongated base member to reflect a relative position of the moveable member with respect to the elongated base member.

9. The measurement device as recited in claim 7, wherein the elongated base member is comprised of a first base member and a second base member that are arranged end-to-end.

10. The measurement device as recited in claim 9, wherein the first base member and the second base member are connected by a hinge.

11. A measurement device for assessing knee movement, comprising:
a first base member;
a second base member arranged end-to-end with the first base member and connected to the first base member by a hinge;
a moveable member configured for movement along and relative to the first and second base members;
indicia on an upper surface of the first base member and the second base member to reflect a relative position of the moveable member with respect to the first and second base members; and
multiple blocks for assessing extension of the knee, said multiple blocks located at a distal end of one of the first base member or the second base member, each of said multiple blocks having a predetermined height, and each of said multiple blocks being configured to be stacked on top of one another.
